(19) United States
(12) Patent Application Publication
(10) Pub. No.: US 2019/0094084 A1
(43) Pub. Date: Mar. 28, 2019

(54) FLUID PRESSURE BASED END EFFECTOR FORCE TRANSDUCER

(71) Applicant: Intuitive Surgical Operations, Inc., Sunnyvale.., CA (US)

(72) Inventors: Charles E. Swinehart, San Jose, CA (US); John Ryan Steger, Sunnyvale, CA (US)

(21) Appl. No.: 16/143,044

(22) Filed: Sep. 26, 2018

Related U.S. Application Data
(60) Provisional application No. 62/563,481, filed on Sep. 26, 2017.

(51) Int. Cl. G01L 1/02; A61B 34/35; A61B 17/29

(52) U.S. Cl. CPC G01L 1/02; A61B 34/35; A61B 2034/301; A61B 2562/0247; A61B 17/29

(57) ABSTRACT

A surgical instrument is provided that includes an elongated shaft; an end effector located at the distal end of the shaft includes first and second jaws having opposing working faces and a pivot axis; at least one of the first and second jaws is mounted to rotatably pivot about the pivot axis. A fluid filled sac includes a first bladder portion and a second bladder portion and a tube portion extending between the first and second bladder portions; the first bladder portion is located at a working face of the first jaw; a sensor is operatively coupled to the second bladder portion to produce a sensor signal indicative of fluid pressure within the fluid filled sac.

### CROSS-REFERENCE TO RELATED APPLICATIONS

This application claims the benefit of priority under 35 U.S.C. § 119(e) to U.S. Provisional Patent Application Ser. No. 62/563,481, filed on Sep. 26, 2017, which is incorporated by reference herein in its entirety.

### BACKGROUND

Minimally invasive medical techniques are intended to reduce the amount of tissue that is damaged during diagnostic or surgical procedures, thereby reducing patient recovery time, discomfort, and deleterious side effects. Teleoperated surgical systems that use robotic technology (so-called surgical robotic systems) may be used to overcome limitations of manual laparoscopic and open surgery. Advances in telepresence systems provide surgeons views inside a patient's body, an increased number of degrees of motion of surgical instruments, and the ability for surgical collaboration over long distances. In manual minimally invasive surgery, surgeons feel the interaction of the instrument with the patient via a long shaft, which eliminates tactile cues and masks force cues. In teleoperation surgery systems, natural force feedback is largely eliminated because the surgeon no longer manipulates the instrument directly. Kinesthetic or force feedback systems typically measure or estimate the forces applied to the patient by the surgical instrument.

### SUMMARY

In one aspect, a surgical instrument is provided that includes an elongated shaft having a proximal end and a distal end. An end effector located at the distal end of the shaft includes first and second jaws having opposing working faces and a pivot axis. At least one of the first and second jaws is mounted to rotatably pivot about the pivot axis between an open position and a closed position. A fluid filled sac includes a first bladder portion and a second bladder portion and a tube portion extending between the first and second bladder portions. The first bladder portion is located at a working face of the first jaw. A sensor is operatively coupled to the second bladder portion to produce a sensor signal indicative of fluid pressure within the fluid filled sac.

### BRIEF DESCRIPTION OF THE DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion. In addition, the present disclosure may repeat reference numerals and/or letters in the various examples. This repetition is for the purpose of simplicity and clarity and does not in itself dictate a relationship between the various embodiments and/or configurations discussed.

FIG. 1 is an illustrative plan view of a minimally invasive teleoperated surgical system.

FIG. 2 is a perspective view of the surgeon's console of the minimally invasive teleoperated surgical system of FIG. 1.

FIG. 3 is a perspective view of a patient-side cart of a minimally invasive teleoperated surgical system of FIG. 1.

FIG. 4 is a perspective view of a surgical instrument used with the minimally invasive teleoperated surgical system of FIG. 1.

FIG. 5 is an illustrative drawing representing a force transducer to convert a fluid pressure caused by an external force to a sensor signal indicative of the magnitude of the force.

FIG. 6A is an illustrative cross-sectional side view of a gripper end effector having the force transducer of FIG. 5 positioned thereon to transduce a force imparted to a working face of an end effector jaw.

FIG. 6B is a top elevation view of an end effector jaw face having a fluid filled first end portion and tube portion of a transducer sac.

FIG. 7A is an illustrative perspective view of a removable force transducer sac having portions thereof embedded within a cap and a collar.

FIG. 7B is an illustrative perspective view of a surgical instrument with the removable sac of FIG. 7A installed.

FIG. 7C is an illustrative cross sectional end view of a shaft having a sensor mounted at a perimeter thereof in alignment with the second end portion of the sac embedded within the collar of FIGS. 7A-7B.

FIG. 8A is an illustrative cross-sectional partially transparent side view of force transducers of FIG. 5 positioned in a wrist portion of a surgical instrument with a single wire anchor.

FIG. 8B is the illustrative partially transparent side view of the surgical instrument and force transducers of FIG. 8A with an external first direction (clockwise) rotation force imparted to the cantilever beam portion.

FIG. 8C is the illustrative partially transparent side view of the surgical instrument and force transducers of FIG. 8A with an external second direction (counter-clockwise) rotation force imparted to the cantilever beam portion.

FIG. 9A is an illustrative cross-sectional partially transparent side view of force transducers of FIG. 5 positioned in a wrist portion of a surgical instrument with a split wire anchor.

FIG. 9B is the illustrative partially transparent side view of the surgical instrument and force transducers of FIG. 9A with an external first direction (clockwise) rotation force imparted to the cantilever beam portion.

FIG. 9C is the illustrative partially transparent side view of the surgical instrument and force transducers of FIG. 8A with an external second direction (counter-clockwise) rotation force imparted to the cantilever beam portion.

FIGS. 10A-10B are illustrative perspective, partially cut away, partially transparent, views of q wrist portion of an end effector with force transducer force transducer to sense rotational force.

### DESCRIPTION OF EMBODIMENTS

### Teleoperated Surgical System

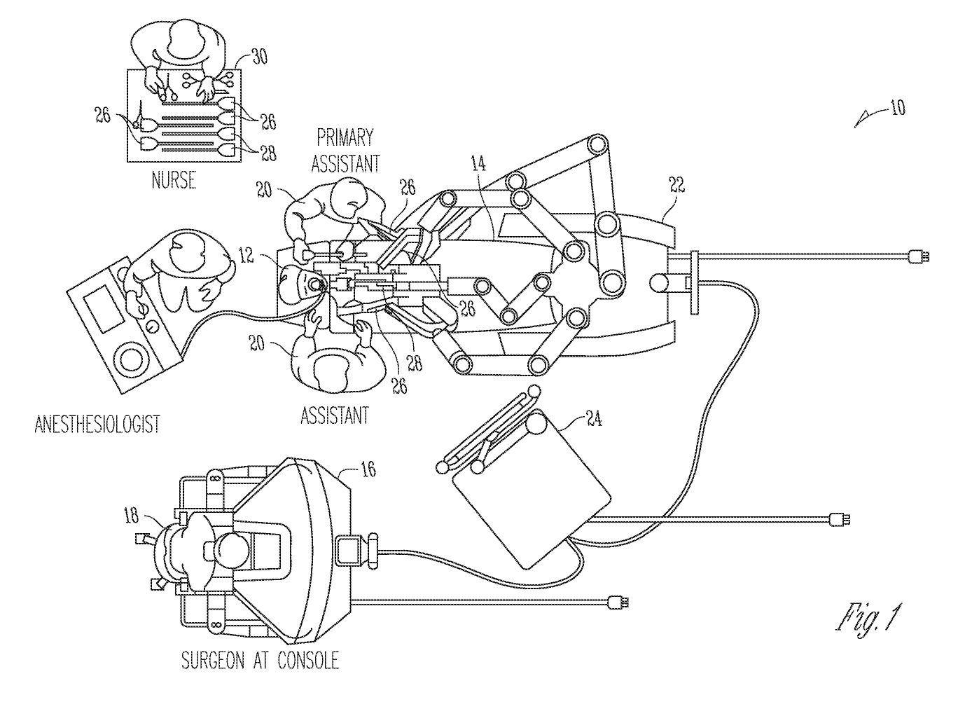

FIG. 1 is an illustrative plan view of a minimally invasive teleoperated surgical system 10 for performing a minimally invasive diagnostic or surgical procedure on a patient 12 who is lying on an operating table 14. The system includes a surgeon's console 16 for use by a surgeon 18 during the procedure. One or more assistants 20 may also participate in the procedure. The minimally invasive teleoperated surgical system 10 further includes one or more patient-side cart 22 and an electronics cart 24. The patient-side cart 22 can manipulate at least one surgical instrument 26 through a minimally invasive incision in the body of the patient 12 while the surgeon 18 views the surgical site through the surgeon's console 16. An image of the surgical site can be obtained by an endoscope 28, such as a stereoscopic endoscope, which may be manipulated by the patient-side cart 22 to orient the endoscope 28. Computer processors located on the electronics cart 24 may be used to process the images of the surgical site for subsequent display to the surgeon 18 through the surgeon's console 16. In some embodiments, stereoscopic images may be captured, which allow the perception of depth during a surgical procedure. The number of surgical instruments 26 used at one time will generally depend on the diagnostic or surgical procedure and the space constraints within the operative site among other factors. If it is necessary to change one or more of the surgical instruments 26 being used during a procedure, an assistant 20 may remove the surgical instrument 26 from the patient-side cart 22, and replace it with another surgical instrument 26 from a tray 30 in the operating room.

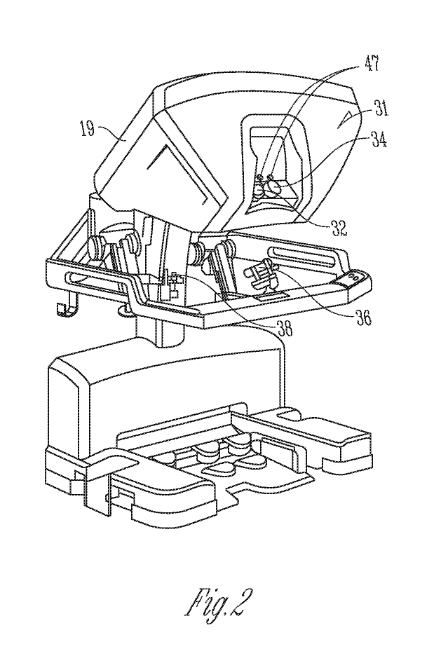

FIG. 2 is a perspective view of the surgeon's console 16. The surgeon's console 16 includes a viewer display 31 that includes a left eye display 32 and a right eye display 34 for presenting the surgeon 18 with a coordinated stereoscopic view of the surgical site that enables depth perception. The console 16 further includes one or more hand-operated control inputs 36 to receive the larger-scale hand control movements. One or more surgical instruments installed for use on the patient-side cart 22 move in smaller-scale distances in response to surgeon 18's larger-scale manipulation of the one or more control inputs 36. The control inputs 36 may provide the same mechanical degrees of freedom as their associated surgical instruments 26 to provide the surgeon 18 with telepresence, or the perception that the control inputs 36 are integral with the instruments 26 so that the surgeon has a strong sense of directly controlling the instruments 26. To this end, position, force, and tactile feedback sensors (not shown) may be employed to transmit position, force, and tactile sensations from the surgical instruments 26 back to the surgeon's hands through the control inputs 36, subject to communication delay constraints.

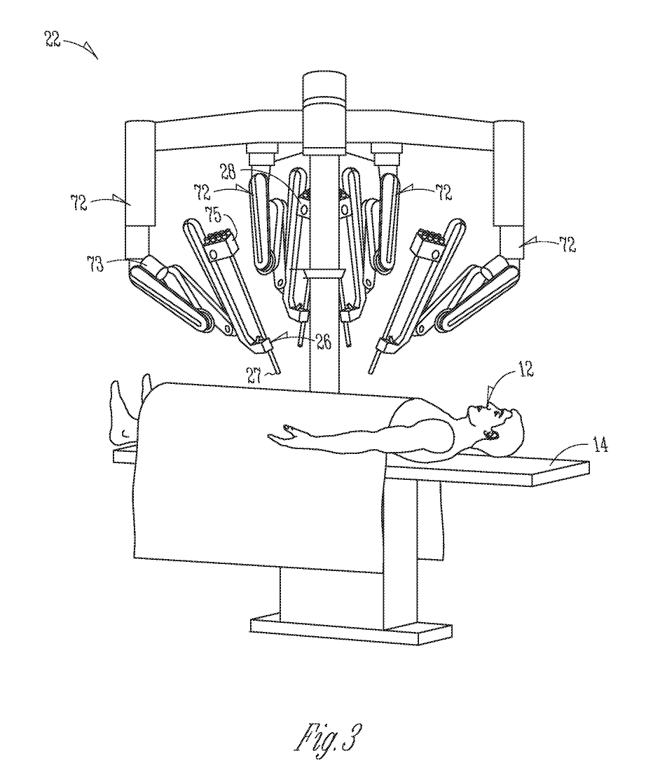

FIG. 3 is a perspective view of a patient-side cart 22 of a minimally invasive teleoperated surgical system 10, in accordance with embodiments. The patient-side cart 22 includes four mechanical support arms 72. A surgical instrument manipulator 73, which includes motors to control instrument motion, is mounted at the end of each support arm assembly 72. Additionally, each support arm 72 can optionally include one or more setup joints (e.g., unpowered and/or lockable) that are used to position the attached surgical instrument manipulator 73 in relation to the patient for surgery. While the patient-side cart 22 is shown as including four surgical instrument manipulators 73, more or fewer surgical instrument manipulators 73 may be used. A teleoperated surgical system will generally include a vision system that typically includes a endoscopic camera instrument 28 for capturing video images and one or more video displays for displaying the captured video images.

In one aspect, for example, individual surgical instruments 26 and cannulas 27 are removably coupled to manipulator 73, with the surgical instrument 26 inserted through the cannula 27. One or more teleoperated actuator motors of the manipulator 73 move the surgical instrument 26 as a whole. The manipulator 73 further includes an instrument carriage 75. The surgical instrument 26 is detachably connected to the instrument carriage 75. In one aspect, the instrument carriage 75 houses one or more teleoperated actuator motors (not shown) inside that provide a number of controller motions that the surgical instrument 26 translates into a variety of movements of an end effector on the surgical instrument 26. Thus, the teleoperated actuator motors in the instrument carriage 75 move only one or more components of the surgical instrument 26 rather than the instrument as a whole. Inputs to control either the instrument as a whole or the instrument's components are such that the input provided by a surgeon or other medical person to the control input (a “master” command) is translated into a corresponding action by the surgical instrument (a “slave” response). A wire cable-based force transmission mechanism or the like is used to transfer the motions of each of the remotely located teleoperated actuator motors to a corresponding instrument-interfacing actuator output located on instrument carriage 75. In some embodiments, the surgical instrument 26 is mechanically coupled to a first actuator motor, which controls a first motion of the surgical instrument such as longitudinal (z-axis) rotation. The surgical instrument 26 is mechanically coupled to a second actuator, which controls second motion of the surgical instrument such as two-dimensional (x, y) motion. The surgical instrument 26 is mechanically coupled to a third actuator, which controls third motion of the surgical instrument such as opening and closing of jaws of an end effector, for example.

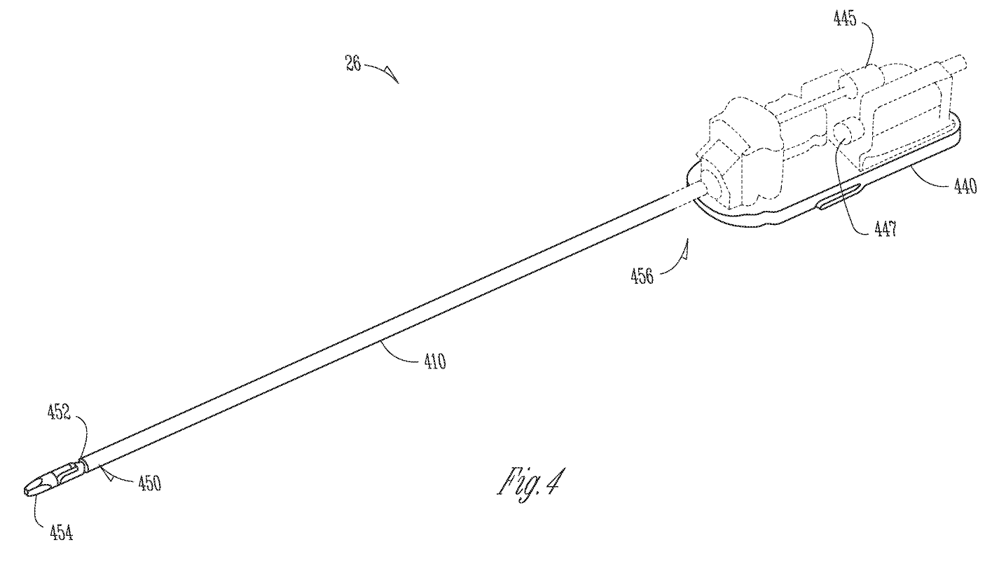

FIG. 4 is a perspective view of a surgical instrument 26, which includes an elongated hollow tubular shaft 410 having a centerline longitudinal axis 411, a distal (first) end portion 450 for insertion into a patient's body cavity and proximal (second) end portion 456 coupled adjacent a control mechanism 440 that includes multiple actuator motors 445, 447 (shown with dashed lines) that exert force upon wire cables coupled to impart motion to the end effector such as opening or closing of jaws and (x, y) wrist motion of a wrist. The surgical instrument 26 is used to carry out surgical or diagnostic procedures. The distal portion 450 of the surgical instrument 26 can provide any of a variety of end effectors 454, such as the forceps shown, a needle driver, a cautery device, a cutting tool, an imaging device (e.g., an endoscope or ultrasound probe), or the like. The surgical end effector 454 can include a functional mechanical degree of freedom, such as jaws that open or close, or a knife that translates along a path or a wrist that may move in x and y directions. In the embodiment shown, the end effector 454 is coupled to the elongated hollow shaft 410 by a wrist 452 that allows the end effector to be oriented relative to the elongate tube centerline axis 411. The control mechanism 440 controls movement of the overall instrument and the end effector at its distal portion.

### End Effector Force Transducer

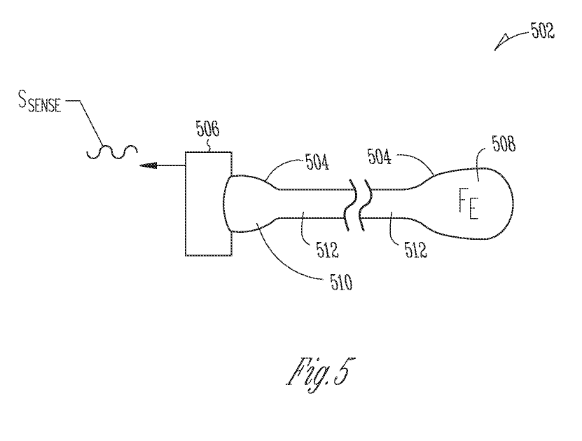

FIG. 5 is an illustrative drawing representing a force transducer 502 to convert a fluid pressure caused by an external force F_{E} to a sensor signal S_{Sense} indicative of the magnitude of the force. Force applied due to pressure equals the Pressure*Area. The force sensor 502 includes a compressible or non-compressible fluid filled sac 504. The partially constrained fluid filled sac 504 contains a fluid having a fluid pressure indicative of magnitude of an external force imparted to the partially constrained sac 508. A sensor 506 is operatively coupled to sense changes in fluid pressure within the sac 504. The sac includes a force receiving first end portion 508 and sensor transducing second end portion 510 and an elongated tube portion 512 extending between them to provide fluid communication between them. The force receiving first end portion 508 may be positioned upon a surgical instrument (not shown) at a location where an external force F_{E} received. The transducing second end portion 510 and the sensor 506 are located where there is sufficient space to house them, within a surgical instrument shaft, for example. The tube 512 couples fluid pressure changes within the sac 504 caused by an external force F_{E}, from the force receiving first end portion 508 to the force transducing second end portion 510. The sensor transducing second portion 510 is operatively coupled to the sensor 506 to convert a change in fluid pressure within the sac 504 to a change in the sensor signal S_{Sense} produced by the sensor 506. In some embodiments, sacs on both ends of the force transducer 502 are constrained to direct the force F_{E} at a surface of the first end portion 508 to the sensor 506 at a surface of the second end portion 510 via the tube portion 510.

The sac 504 may be formed of a flexible material such as Thermo Plastic Elastomer or Silicone Rubber etc. or a deformable material such as mylar, for example. The fluid within the sac 504 may include an incompressible fluid such as water or other biologically safe fluid. The fluid within the sac 504 may include a compressible fluid such as Nitrogen, carbon dioxide or other biologically safe gas. In some embodiments, the force receiving first end portion 508 is configured as a first bladder that has a wider diameter dimension than the tube 512, to provide an increased surface area to receive the external force F_{E} imparted through contact with anatomical tissue (not shown), for example. (The first end portion may be referred to herein interchangeably as the first bladder.) The narrower dimension tube 512 is less susceptible to breaking due to rough treatment during a surgery or cleaning than an optical fiber or wires. In alternative embodiment, the first end portion 508, the second end portion 510 and the tube 512 have identical diameters, and both ends 508, 510 of the force transducer 502 are constrained to direct the force F_{E} at a surface of the first end portion 508 to the sensor 506 at a surface of the second end portion 510 via the tube portion 510.

### Operative Coupling Between Fluid Pressure and Sensor Signal

The sensor transducing second end portion 510 is operatively coupled to the sensor 506 to convert a change in fluid pressure within the sac 504 to a change in the sensor signal S_{Sense} produced by the sensor 506 located within the shaft 410. The sensor 506 may include a MEMS pressure sensor and the sensor signal S_{Sense} includes an electrical signal in which a change in force imparted by the second end portion 510 upon a pressure sensing surface of the MEMS pressure sensor causes change in an electrical signal produced by the MEMS device. Alternatively, the sensor 506 may include a fiber Bragg grating (FBG) pressure sensor and the sensor signal S_{Sense} includes an optical signal in which a change in force imparted by the second end portion 510 upon a pressure sensing surface causes change in an optical signal. As another alternative, the sensor 506 may include an optical reflectance based displacement sensor and the sensor signal S_{Sense} includes a light reflectance signal that produces a light reflectance signal that is indicative of a change in light reflectance due to displacement of a surface of a sensor transducing portion facing the sensor due to a change in force imparted by the second end portion 510 due to a change in fluid pressure. Yet another alternative, the sensor 506 may include Hall effect sensor and the sensor signal S_{Sense} includes a magnetic signal that is indicative of a change in a magnetic field caused by displacement of a surface of the sensor transducing portion facing the sensor due to a change in force imparted by the second end portion 510 due to a change in fluid pressure. As still another alternative, the sensor 506 may include capacitive sensor and the sensor signal S_{Sense} includes an electrical signal that is indicative of a change in capacitance caused by displacement of a surface of the sensor transducing portion facing the sensor due to a change in force imparted by the second end portion 510 due to a change in fluid pressure.

### End Effector Jaw Force Transducers

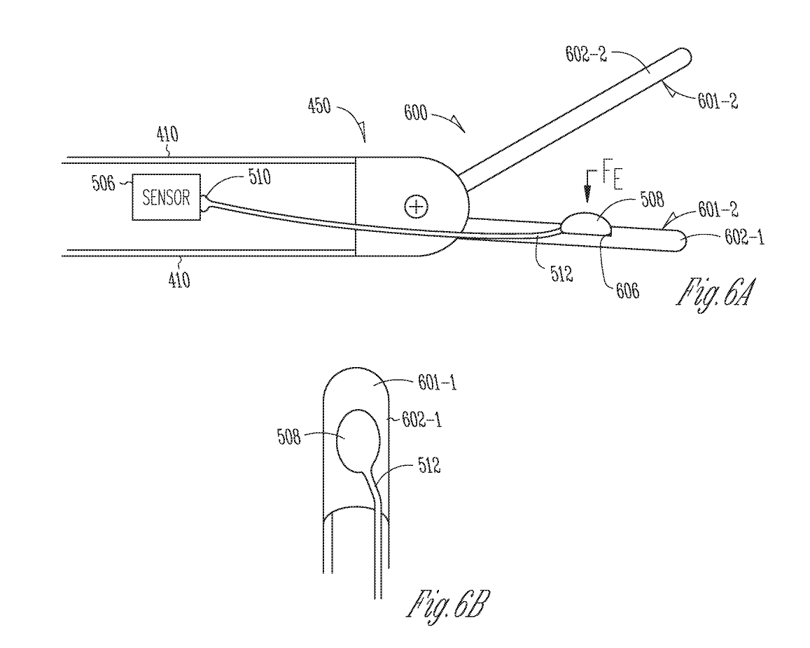

FIG. 6A is an illustrative cross-sectional side view of a gripper end effector 600 having the force transducer 502 of FIG. 5 positioned thereon to transduce a force imparted to a working face 601-1 of a first end effector jaw 602-2. FIG. 6B is a top elevation view of the working face 601-1 of the first end effector jaw 602-1 having a fluid filled first end portion 508 and a portion of the tube 512 disposed thereon. The gripper end effector 600 is located at a distal end portion 450 of a hollow surgical instrument shaft 410 and may include opposed facing first and second jaws 602-1, 602-1 that may grasp anatomical tissue (not shown) between them, for example. Each jaw has a working surface 601-1, 601-2 that faces an opposite-facing jaw that contacts tissue grasped between the jaws. The grasping of tissue between the jaws may impart an external force F_{E} upon the working surface 601-1 of the first jaw 602-1 and upon a first end portion 508 of the force transducer 502 located thereon.

More particularly, a force receiving first end portion 508 of the force transducer sac 504 is within a recess 606 formed in the working surface 601-1 of the first jaw 602-1. The first end portion 508 of the sac 504 is configured as a fluid filled first bladder that has a larger diameter cross-section (or width) dimension than the tube 512, to provide a wider surface area to the receive external force F_{E}. The first end portion 508 has a thickness (or height) dimension that is greater than the depth of the recess 606 so that it protrudes outwardly from the recess 606 to contact anatomical tissue (not shown) that may be gripped between the jaws 602-1, 602-2. In some embodiments, the first end portion 508 is removably secured to the first jaw face 601-1 by a snug interfit with the walls of the recess 606 sufficient to hold the first end portion 508 in place. Alternatively, a flexible diaphragm (not shown) may fit abound the first jaw 602-1 and overlay the first end portion 508 with the first end portion 508 therebetween to hold the first end portion 508 in place within the recess 606. A sensor transducing second end portion 510 of the sac 504 and a sensor 506 are disposed within the hollow shaft 410 proximal to the end effector 600. A tube portion 512 of the first force transducer sac 504 extends along the shaft 410 between the first and second end portions 508, 510 of the sac 504. Alternatively, the sensor 506 may be disposed at a proximal end 456 of the shaft 410 or outside (not shown) the shaft 410, for example In operation, as the jaws 602-1, 602-2 squeeze anatomical tissue (not shown) between them, and an external force F_{E} imparted by the tissue squeezes the first end portion 508 (the first bladder 508), increasing fluid pressure within it and within the tube 512. The tube 512 communicates the increased pressure to the sensor transducing second portion 510 to cause the sensor 506 to produce a sensor signal S_{Sense} indicative of the change in fluid pressure within the fluid filled sac 504.

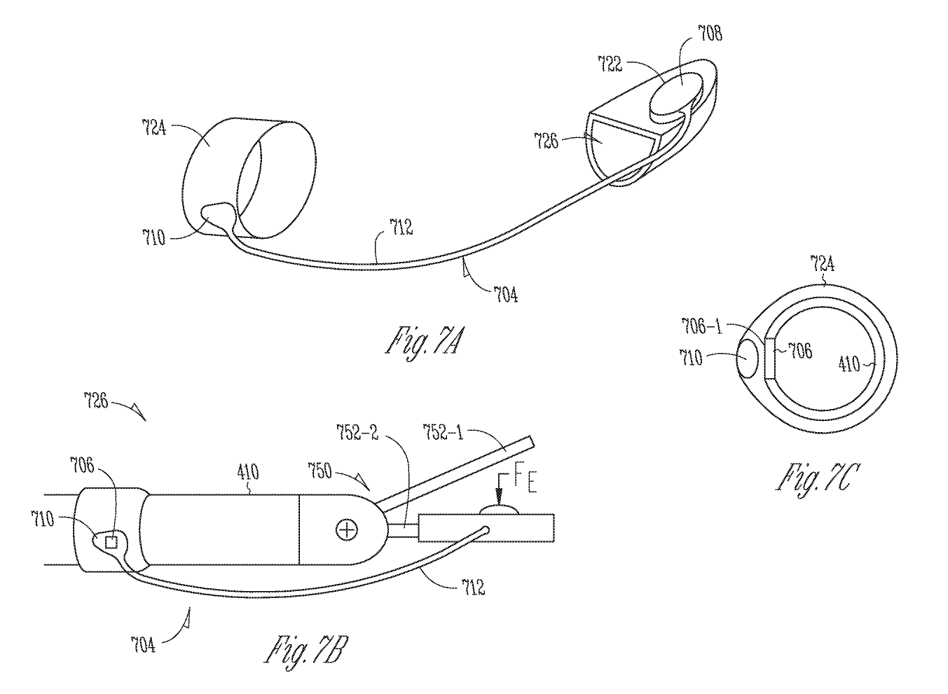

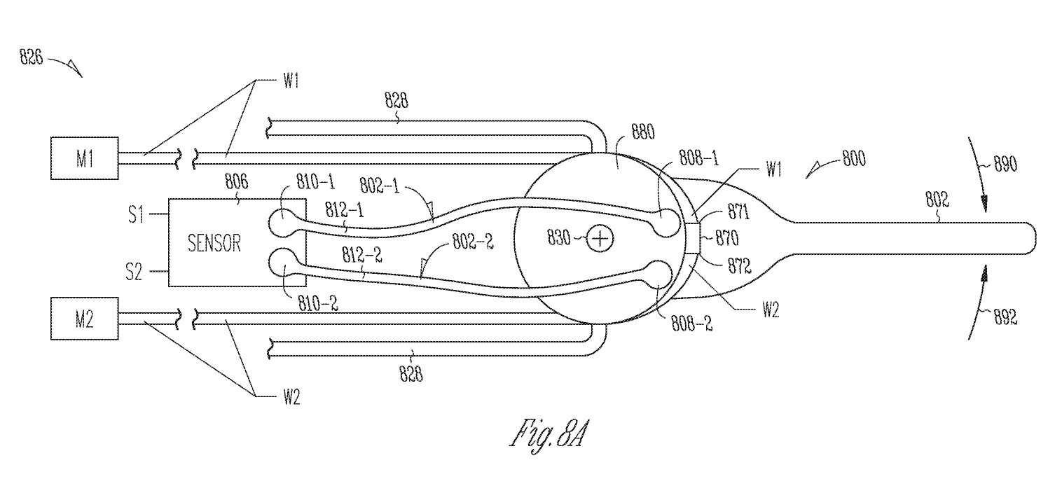

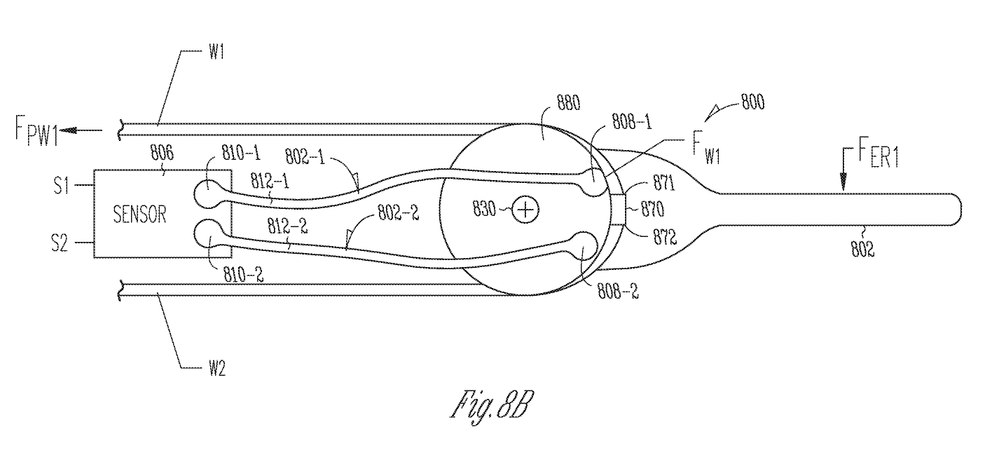

FIG. 7A-7B are illustrative perspective views of a removable force transducer sac 702 having portions thereof embedded within a cap 722 and a collar 724 (FIG. 7A) and a surgical instrument 726 having a gripper end effector 750 with the removable sac 502 installed thereon. (FIG. 7B) A flexible fluid filled first end portion 708 of the sac 704 is integrally formed with the cap 722, which is sized to snugly fit over a first 752-1 of the gripper end effector 750. The gripper end effector 749 includes first and second opposed facing jaws 752-1, 752-2. The cap 722 is sized to removably fit over the first end effector jaw 752-1 with the force receiving end portion 708 of the sac 704 disposed to receive an external force F_{E} imparted to the working surface of the first jaw 752-1. The cap 722 includes defines an inner hollow space 726 open at one end to receive insertion of the first jaw 752-1. The first end portion 708 of the sac 704 embedded within the cap 722 overlays a working surface of the first jaw 752-1 that faces the second jaw 752-2. A fluid filled second end portion 710 of the sac 704 is integrally formed within a collar 724 to removably fit snugly about an exterior of the surgical instrument shaft 410. A fluid filled tube 712 extends along an outside surface of the surgical instrument shaft 410 between the fluid filled force receiving first end portion 708 embedded within the cap 722 and the second end portion 710 embedded within the collar 724 to provide fluid communication between them.

FIG. 7C is an illustrative axial cross sectional view of a shaft having a sensor 706 mounted at a perimeter thereof operatively coupled to the fluid filled sac 704 embedded within the collar 724 of FIGS. 7A-7B disposed about the shaft 410. The sensor 706 is operatively coupled to produce a sensor signal S_{Sense} indicative of fluid pressure within the fluid filled sac 704. An external force F_{E} imparted to the first end portion 708 may cause an increase in fluid pressure that is communicated via tube 712 to the second end portion 710, which is operatively couple to the sensor 706. The sensor 706 is operatively coupled to produce a change in the sensor signal S_{Sense} in response to a change of fluid pressure within the fluid filled sac 704. The shaft 410 has a circular cross-section. The collar 724 is formed of a flexible material such as Thermo Plastic Elastomer or Silicone Rubber etc. sized to snugly fit about a portion of the shaft where the sensor 706 is located and to permit slidable alignment of the second end portion 710 and the sensor 706 and. The second end portion 710 of the sac 704 is disposed at a sub-portion of the collar 724 that is large enough to make operative contact with a pressure sensing surface portion 706-1 of the sensor 706 such that a change in fluid pressure within the fluid filled sac 704 is imparted to the pressure sensing surface portion 706-1 of the sensor 706. In some embodiments, the second end portion 710 may be configured as a second bladder that has a wider diameter dimension than the tube 712, to provide an increased surface area to transduce force to the sensor 706, for example. (The second end portion may be referred to herein interchangeably as the second bladder.) The removable force transducer sac 704 may disposable after one or few surgeries while the sensor may be reused in more surgeries, for example.

### End Effector Rotational Force Transducers

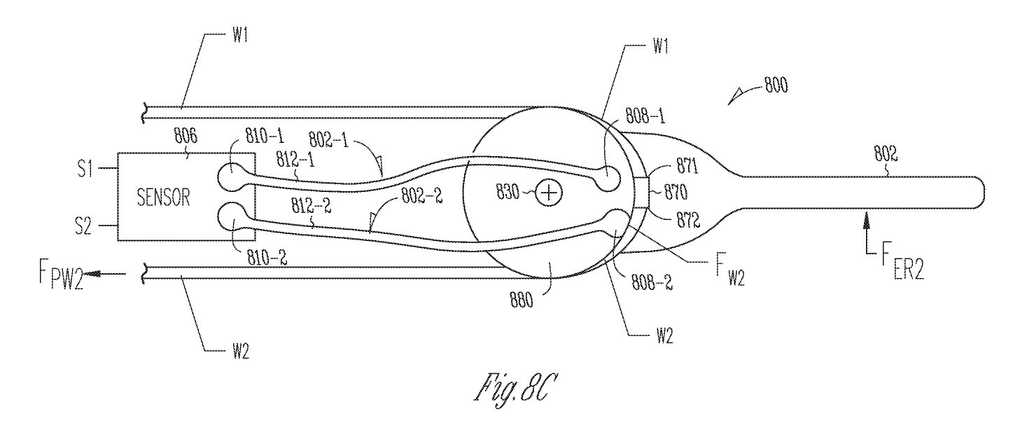

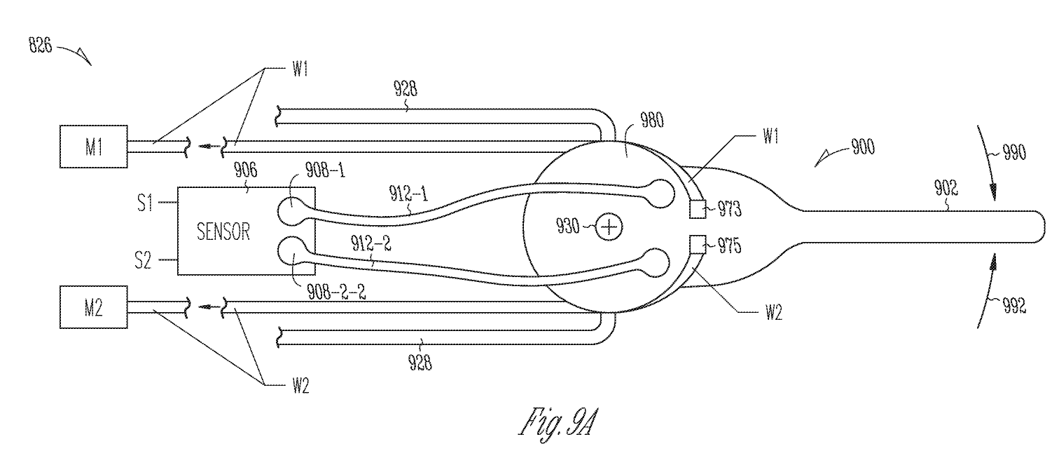

FIG. 8A is an illustrative cross-sectional partially transparent side view of first and second force transducers 802-1, 802-2 are positioned in a wrist portion of a surgical instrument 826 with a single wire anchor 870 to transduce a rotational force imparted to an end effector 800. The end effector 800 includes a cantilever beam portion 802 such as a jaw or a blade integrally secured to and depending from a pulley 880 that is rotatably mounted between arms of a clevis (not shown) disposed at the distal end of a hollow surgical instrument shaft 828 for rotation about a pulley axis 830. The cantilever beam portion 802 may include opposed facing jaws with or without associated sensors (not shown) to grasp anatomical tissue between them, for example. Alternatively, for example, the cantilever beam portion 802 may include a dissecting blade (not shown) to cut through anatomical tissue, for example. A first force transducer 802-1 is positioned within the surgical instrument 826 to transduce a first external rotational force imparted to the cantilever beam portion 802 in a first (clockwise) direction (indicated by arrow 890) about the pulley axis 830. A second force transducer 802-2 is positioned within the surgical instrument 826 to transduce a second external rotational force imparted to the cantilever beam portion 802 in a second (counter-clockwise) direction (indicated by arrow 892) about the pulley axis 830. The first force transducer 802-1 includes a first and second fluid filled end portions 808-1, 810-1 and a fluid-filled tube 812-1 to communicate fluid pressure change between them. The second force transducer 802-2 also includes a first and second fluid filled end portions 802-2, 808-10 and a fluid-filled tube 812-2 to communicate fluid pressure change between them. The second end portions 808-1, 808-2 of both the first and second force transducers 802-1, 802-2 are coupled to a shared sensor 806, which may be disposed within the hollow surgical instrument shaft 828.

First and second wires W1, W2 extend within the shaft 828 along opposite sides of the second shaft 828 to control rotational position of the pulley 880, and of the cantilever beam portion 802 depending therefrom, about the pulley axis 830. An anchor structure 870 is secured to a face of the end effector 800 adjacent to an outer perimeter of the pulley 880. A distal end of the first wire W1 is secured to a first side 871 of the anchor structure 870 and extends within a circumferential groove (not shown) in an outer edge of the pulley 880 between the first side 871 of the anchor structure 870 and the shaft 828. A distal end of the second wire W2 is secured to a second side 872 of the anchor structure 870 and extends within a circumferential groove (not shown) in an outer edge of the pulley 880 between the second side 872 of the anchor 870 and the shaft 828. A first actuator motor M1 may impart a first proximal direction force upon the first wire W1 coupled to the anchor first side 871 of the anchor 870 to pull the pulley 880 in a second (counter-clockwise) direction. A second actuator motor M2 may impart a second proximal direction force upon the second wire W2 coupled to the second side 872 of the anchor 870 to pull the pulley 880 in a second (clockwise) rotation.

The first fluid filled end portion 808-1 of the first force transducer 802-1 protrudes from a surface of end effector 800 at a radial distance from the pulley axis greater than the pulley diameter and close enough to a location where the first wire W1 physically connects with the first side 871 of the anchor 870 that tensioning of the first wire W1 by a proximal direction force imparted to the first wire W1 causes it to contact and exert an external force upon the first fluid filled end portion 808-1 of the first force transducer 802-1. Alternatively, or in addition, the anchor structure 870 itself may contact and apply an external force upon the first fluid filled end portion 808-1 when the first wire W1 is tensioned.

Similarly, the second fluid filled end portion 808-2 of the second force transducer 802-2 protrudes from a surface of end effector 800 at a radial distance from the pulley axis greater than the pulley diameter and close enough to a location where the second wire W2 physically connects with the second side 872 of the anchor 870 that tensioning of the second wire W2 by a proximal direction force imparted to the second wire W2 causes it to contact and exert an external force upon the second fluid filled end portion 808-2 of the second force transducer 802-2. Alternatively, or in addition, the anchor structure 870 itself may contact and apply an external force upon the second fluid filled end portion 808-2 when the second wire W2 is tensioned.

FIG. 8B is the illustrative partially transparent side view of the surgical instrument 826 and force transducers 802-1, 802-2 of FIG. 8A with an external first direction (clockwise) rotation force F_{ER1} imparted to the cantilever beam portion 802. In some embodiments, the first actuator motor M1 reacts to the first direction (clockwise) force F_{ER1} imparted to the cantilever beam portion 802 by imparting a first proximal direction rotational force F_{PW1} to the first wire which urges rotation of the pulley 880 in the second (counter-clockwise) direction so as to resist the external first direction force F_{ER1} upon the cantilever beam portion 802. The first actuator motor force F_{PW1} imparted to the first wire W1 increases tension in the first wire W1. The increased tension in the first wire W1 removes slack from the first wire W1 causing the first wire W1 and/or the first side 871 of the anchor 870 to make contact with and to impart an external force F_{W1} upon the first fluid filled end portion 808-1 of the first force transducer 802-1. The increased pressure is communicated via to the sensor 806 via the tube first tube 812-1 and the second end portion 810-1, which produces a first sensor signal S1 indicative of the increased pressure.

FIG. 8C is the illustrative partially transparent side view of the surgical instrument 826 and force transducers 802-1, 802-2 of FIG. 8A with an external second direction (counter-clockwise) rotation force F_{ER2} imparted to the cantilever beam portion 802. In some embodiments, the second actuator motor M2 reacts to the second direction (counter-clockwise) force F_{ER2} imparted to the cantilever beam portion 802 by imparting a second proximal direction rotational force F_{PW2} to the second wire which urges rotation of the pulley 880 in the first (counter-clockwise) direction so as to resist the external second direction force F_{ER2} upon the cantilever beam portion 802. The second actuator motor force F_{PW2} imparted to the second wire W2 increases tension in the second wire W2. The increased tension in the second wire W2 removes slack from the second wire W2 causing the second wire W2 and/or the second side 872 of the anchor 870 to make contact with and to impart an external force F_{W2} upon the second fluid filled end portion 808-2 of the second force transducer 802-2. The increased pressure is communicated via to the sensor 806 via the second tube 812-2 and the second end portion 810-2, which produces a second sensor signal S2 indicative of the increased pressure.

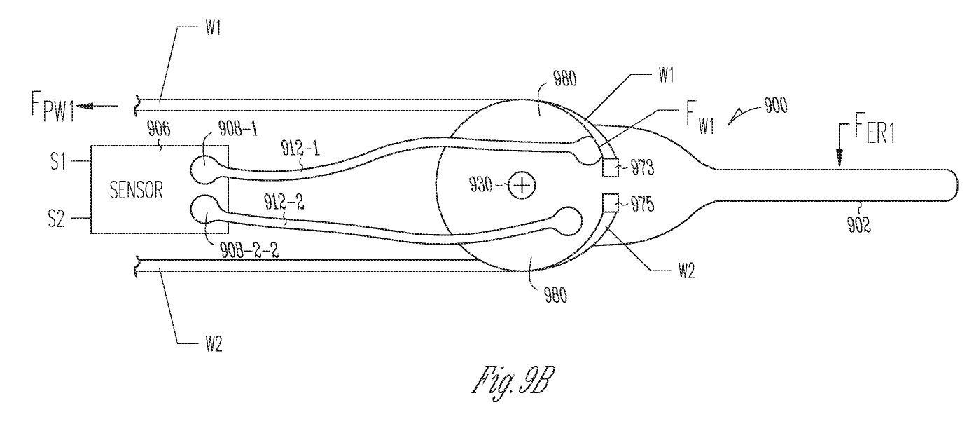

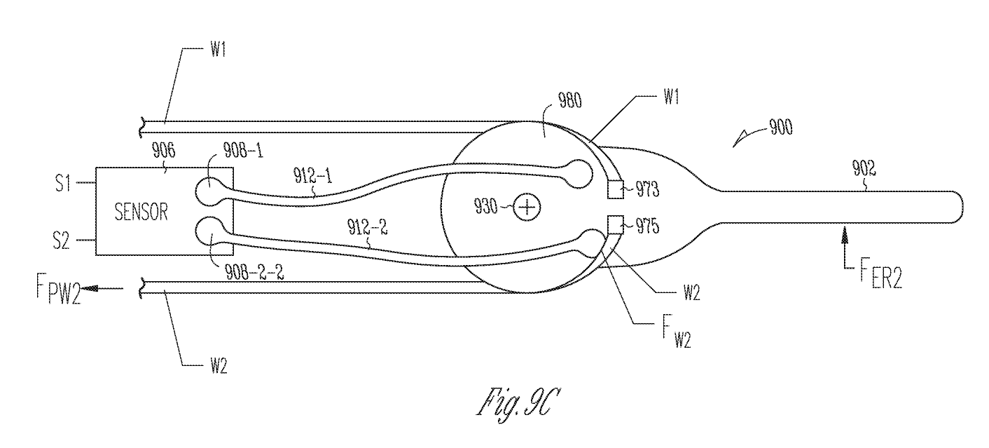

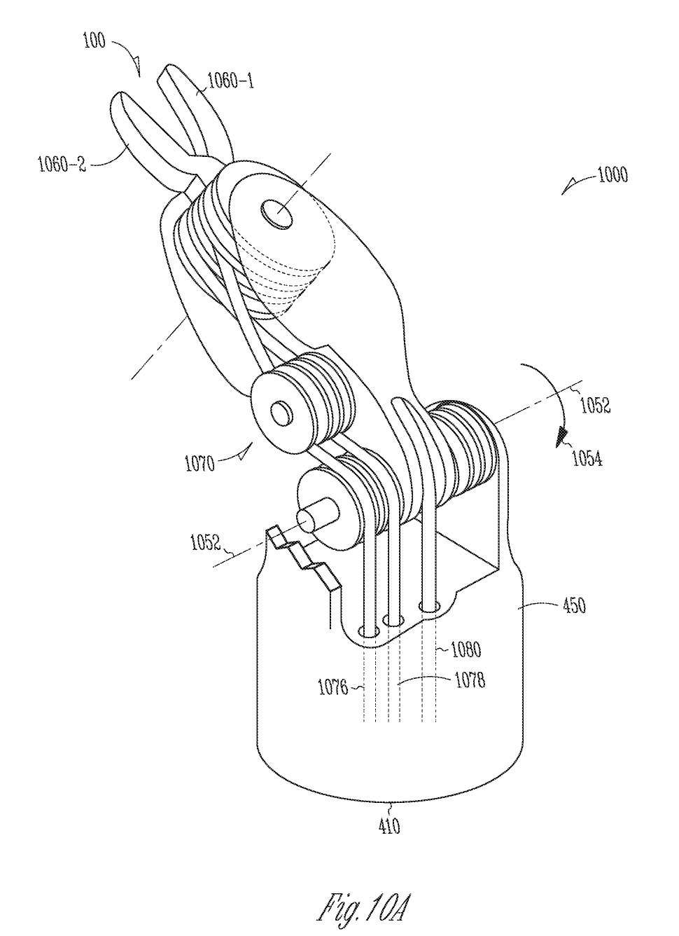

FIG. 9A is an illustrative cross-sectional partially transparent side view of first and second force transducers 902-1, 902-2 of positioned in a wrist portion of a surgical instrument 926 with a split wire anchor 973, 975 to transduce a rotational force imparted to an end effector 900. A first split anchor portion 973 and a second split anchor portion 975 are spaced apart from each other and disposed at a radial distance from the pulley axis greater than the pulley diameter. Certain portions of the split wire anchor embodiment and of the single anchor embodiment that are similar are referenced with identical reference numbers and will be understood from the above explanation and will not explained again.

A first fluid filled end portion 908-1 of the first force transducer 902-1 protrudes from a surface of end effector 900 at a radial distance from the pulley axis greater than the pulley diameter and close enough to a location where the first wire W1 physically connects with a first split anchor 973 that tensioning of the first wire W1 by a proximal direction force imparted to the first wire W1 causes it to contact and exert an external force upon the first fluid filled end portion 908-1 of the first force transducer 902-1. Alternatively, or in addition, the first anchor portion 973 itself may contact and apply an external force upon the first fluid filled end portion 908-1 when the first wire W1 is tensioned.

Similarly, the second fluid filled end portion 908-2 of the second force transducer 902-2 protrudes from a surface of end effector 900 at a radial distance from the pulley axis greater than the pulley diameter and close enough to a location where the second wire W2 physically connects with the second split anchor portion 975 that tensioning of the second wire W2 by a proximal direction force imparted to the second wire W2 causes it to contact and exert an external force upon the second fluid filled end portion 908-2 of the second force transducer 902-2. Alternatively, or in addition, the second anchor portion 975 itself may contact and apply an external force upon the second fluid filled end portion 908-2 when the second wire W2 is tensioned.

FIG. 9B is the illustrative partially transparent side view of the surgical instrument 926 and force transducers 902-1, 902-2 of FIG. 9A with an external first direction (clockwise) rotation force F_{ER1} imparted to the cantilever beam portion 902. In some embodiments, the first actuator motor M1 reacts to the first direction (clockwise) force F_{ER1} imparted to the cantilever beam portion 902 by imparting a first proximal direction rotational force F_{PW1} to the first wire which urges rotation of the pulley 980 in the second (counter-clockwise) direction so as to resist the external first direction force F_{ER1} upon the cantilever beam portion 902. The first actuator motor force F_{PW1} imparted to the first wire W1 increases tension in the first wire W1. The increased tension in the first wire W1 removes slack from the first wire W1 causing the first wire W1 and/or the first split anchor 973 to make contact with and to impart an external force F_{W1} upon the first fluid filled end portion 908-1 of the first force transducer 902-1. The increased pressure is communicated via to the sensor 906 via the tube first tube 912-1 and the second end portion 910-1, which produces a first sensor signal S1 indicative of the increased pressure.

FIG. 9C is the illustrative partially transparent side view of the surgical instrument 926 and force transducers 902-1, 902-2 with an external second direction (counter-clockwise) rotation force F_{ER2} imparted to the cantilever beam portion 902. In some embodiments, the second actuator motor M2 reacts to the second direction (counter-clockwise) force F_{ER2} imparted to the cantilever beam portion 902 by imparting a second proximal direction rotational force F_{PW2} to the second wire which urges rotation of the pulley 980 in the first (counter-clockwise) direction so as to resist the external second direction force F_{ER2} upon the cantilever beam portion 902. The second actuator motor force F_{PW2} imparted to the second wire W2 increases tension in the second wire W2. The increased tension in the second wire W2 removes slack from the second wire W2 causing the second wire W2 and/or the split anchor 975 to make contact with and to impart an external force F_{W2} upon the second fluid filled end portion 908-2 of the second force transducer 902-2. The increased pressure is communicated via to the sensor 906 via the second tube 912-2 and the second end portion 910-2, which produces a second sensor signal S2 indicative of the increased pressure.

### Calibration and External Force Determination

During a calibration procedure, one or more actuator motors impart several different rotational calibration forces to each of the first and second wires. The sensor produces a corresponding sensor calibration signal value in response to each imparted calibration force, which is stored in electronic memory storage (not shown) During operation, in which an actuator motor produces a given rotational force to resist an external rotational force imparted to the end effector, a magnitude of the external force imparted to the end effector can be determined based upon a difference between a stored calibration sensor signal value corresponding to the given rotational force and a sensor signal value produced in response to the external force.

### Example End Effector Rotational Force Transducer Embodiment

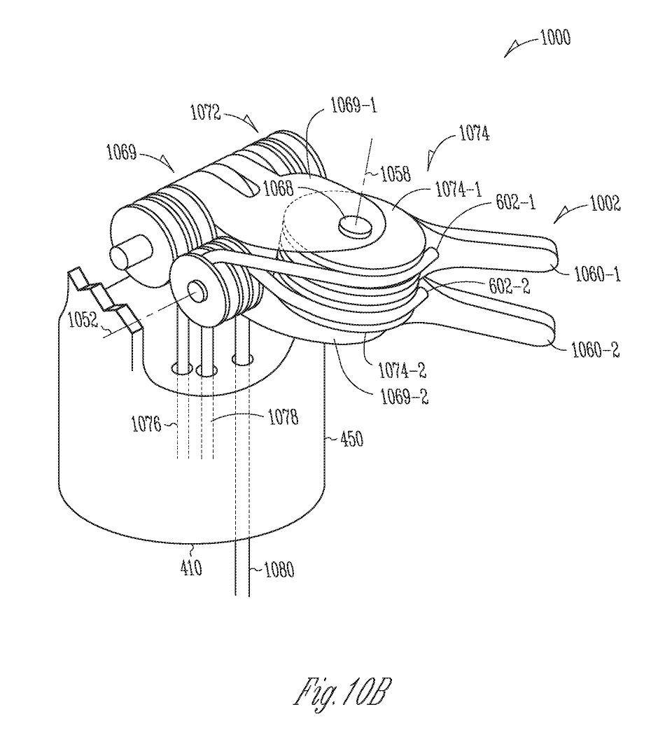

FIGS. 10A-10B are illustrative perspective, partially cut away, views of a pivotable wrist portion 1000 a first position (FIG. 10A) and a second position (FIG. 10B) that mounts an articulable jaw end effector 1002 that includes first and second jaws 1060-1, 1060-2. The wrist portion 1000 is mounted at a distal wrist portion 450 of a surgical instrument shaft 410. The wrist portion 1000 includes a first pulley set 1070, a second pulley set 1072, and a third pulley set 1074 set to guide first, second and third cable segments 1076, 1078, 1180 that extend from within the shaft 410 and about the pulley sets. The wire ropes 1076, 1078, 1080 are used in combination to cause the wrist portion 452 to pivot about a first axis 1052 as indicated by arrow 1054, for example. The cables 1076, 1078, 1080 also are used in combination to cause the end effector portion 1002 of the wrist portion 1000 to pivot about a second axis 1058.

The first jaw 1060-1 is integrally secured to and depends from a first pulley 1074-1 of the third pulley set 1074. The second jaw 1060-2 is integrally secured to and depends from a second pulley 1074-2 of the third pulley set 1074. The first and second pulleys 1074-1, 1074-2 of the third set 1074, which are mounted on an axel 1058 between opposed arms 1069-1, 1069-2 (shown transparent) of a first clevis 1069 for rotation about the second axis 1058.

A first fluid filled force receiving first portion 602-1 of a first force transducer is shown disposed at an interface of the first cable 1076 and the first jaw 1060-1. A fluid filled force receiving first portion 602-2 of a second force transducer, is shown disposed at an interface of the second cable 1078 and the second jaw 1060-2. As will be understood from the explanation above, the fluid filled force receiving first portion 602-1 is disposed close enough to the first cable 1076 such that increased tension upon first cable 1076 may stiffen the first cable 1076 to impart increased force upon the fluid filled force receiving first portion 602-1, which causes a sensor (not shown) to produce a sensor signal value indicative of the increased pressure. Increased tension upon first cable 1076 also may stretch and straighten the first cable, which may contribute to an increased force upon the fluid filled force receiving first portion 602-1 of the first force transducer. Likewise, the fluid filled force receiving first portion 602-2 is disposed close enough to the second cable 1078 such that increased tension upon second cable 1078 may stiffen the second cable 1078 to impart increased force upon the fluid filled force receiving first portion 602-2 of the second force transducer, which causes the sensor (not shown) to produce a sensor signal value indicative of the increased pressure. Increased tension upon second cable 1078 also may stretch and straighten the second cable, which may contribute to an increased force upon the fluid filled force receiving first portion 602-2 of the first force transducer. Additional details of an embodiment of the example wrist portion 1000 are provided in U.S. Pat. No. 6,394,998, entitled, “Surgical Tools for Use in Minimally Invasive Telesurgical Applications”.

Although illustrative embodiments have been shown and described, a wide range of modification, change and substitution is contemplated in the foregoing disclosure and in some instances, some features of the embodiments may be employed without a corresponding use of other features. One of ordinary skill in the art would recognize many variations, alternatives, and modifications. Thus, the scope of the disclosure should be limited only by the following claims, and it is appropriate that the claims be construed broadly and in a manner consistent with the scope of the embodiments disclosed herein. The above description is presented to enable any person skilled in the art to create and use a wire rope with enhanced wire wrap. Various modifications to the embodiments will be readily apparent to those skilled in the art, and the generic principles defined herein may be applied to other embodiments and applications without departing from the spirit and scope of the invention. In the preceding description, numerous details are set forth for the purpose of explanation. However, one of ordinary skill in the art will realize that the invention might be practiced without the use of these specific details. In other instances, well-known processes are shown in block diagram form in order not to obscure the description of the invention with unnecessary detail. Identical reference numerals may be used to represent different views of the same or similar item in different drawings. Thus, the foregoing description and drawings of embodiments in accordance with the present invention are merely illustrative of the principles of the invention. Therefore, it will be understood that various modifications can be made to the embodiments by those skilled in the art without departing from the spirit and scope of the invention, which is defined in the appended claims.

What is claimed is:

1. A surgical instrument comprising:
an elongated shaft including a proximal end and a distal end;
an end effector located at the distal end of the shaft and including first and second jaws having opposing working faces and a pivot axis, wherein at least one of the first and second jaws is mounted to rotatably pivot about the pivot axis between an open position and a closed position;
a fluid filled sac including a first bladder portion and a second bladder portion and a tube portion extending between the first and second bladder portions;
wherein the first bladder portion is located at a working face of the first jaw;
a sensor operatively coupled to the second bladder portion produce a sensor signal indicative of fluid pressure within the fluid filled sac.

2. The surgical instrument of claim 1,
wherein the working face of the jaw defines a recess sized to provide a snug interfit with the first bladder portion.

3. The surgical instrument of claim 1,
wherein the first bladder portion is disposed upon the working surface of the jaw; further including:
a flexible diaphragm fit about the first jaw and the first bladder portion to hold the first bladder portion in place at the working face of the first jaw.

4. The surgical instrument of claim 1,
wherein the sensor is disposed within the elongated shaft.

5. The surgical instrument of claim 1,
wherein the first bladder portion has a wider diameter than the tube portion.

6. A force transducer for use with a surgical instrument that includes a shaft and a gripper end effector at a distal end thereof, comprising:
a jaw cap configured to snugly fit over a jaw of the gripper end effector;
a collar configured to snugly fit about the shaft;
a fluid filled sac including,
a first fluid filled bladder disposed upon a jaw cap;
a second fluid filled bladder disposed upon the collar; and
a fluid filled tube providing fluid communication between the first and second fluid filled bladders.

7. The force transducer of claim 6 further including:
a sensor disposed at the shaft configured for operatively coupling with the second fluid filled bladder to produce a sensor signal indicative of fluid pressure within the sac while the collar is fit about the shaft.

8. The force transducer of claim 6,
wherein the second fluid filled bladder disposed upon a sub-portion of the collar that is large enough for operative coupling with the sensor.

9. The force transducer of claim 6,
wherein the second fluid filled bladder is disposed upon a sub-portion of the collar that is large enough for operative coupling with the sensor; further including:
a sensor disposed at the shaft a perimeter of the shaft configured for alignment with the sub-portion of the collar and for operatively coupling with the second fluid filled bladder to produce a sensor signal indicative of fluid pressure within the sac while the collar is fit about the shaft.

10. A surgical instrument comprising:
an elongated shaft including a proximal end and a distal end;
an end effector located at the distal end of the shaft and including first and second jaws having opposing working faces and a pivot axis, wherein at least one of the first and second jaws is mounted to rotatably pivot about the pivot axis between an open position and a closed position;
a transducer sac that includes,
a jaw cap disposed about at least a portion of the first jaw that includes a first fluid filled bladder portion disposed over at least a portion of a working face of the first jaw;
a collar disposed about the shaft that includes a second fluid filled bladder portion;
a fluid filled tube portion integrally formed with the first and second bladder portions and providing fluid communication between the first and second fluid filled bladder portions; and
a sensor operatively coupled to the second bladder portion to produce a sensor signal indicative of fluid pressure within the transducer sac.

11. The surgical instrument of claim 10,
wherein the sensor is disposed at a perimeter of the shaft.

12. The surgical instrument of claim 10,
wherein the first bladder portion has a wider diameter than the tube portion.

13. The surgical instrument of claim 10,
wherein the tube portion extends outside the shaft between the first and second bladder portions.

14. A surgical instrument comprising:
an elongated hollow shaft including a proximal end and a distal end;
a pulley rotatably mounted at the distal end of the shaft for rotation about a pivot axis;
a cantilever end effector extending from the pulley to rotate with the pulley about the pivot axis;
a first wire extending within the shaft and engaging a first perimeter portion of the pulley and having a distal end secured to the end effector;
a first actuator to provide the proximal direction force to the first wire;
a first fluid filled sac including a first distal bladder portion positioned at a surface of the cantilever end effector to receive a force imparted by at least one of the first wire and a first wire anchor by contact with the at least one of the first wire and the first wire anchor, while the first actuator imparts the proximal direction force to the first wire; and
a first sensor operatively coupled to produce a first sensor signal indicative of fluid pressure within the first fluid filled sac.

15. The surgical instrument of claim 14,
wherein the first fluid filled sac further includes a first proximal bladder portion and a first tube portion extending between the distal and proximal bladder portions; and
wherein the first sensor is operatively coupled to the proximal bladder portion to produce the first sensor signal indicative of fluid pressure within the first fluid filled sac.

16. The surgical instrument of claim 14,
wherein the sensor is disposed within the elongated shaft.

17. The surgical instrument of claim 14,
wherein the first bladder portion has a wider diameter than the tube portion.

18. The surgical instrument of claim 14 further including:
a second wire extending within the shaft and engaging a second perimeter portion of the pulley and having a distal end secured to the end effector;
a second actuator to provide the proximal direction force to the second wire;
a second fluid filled sac including a second distal bladder portion positioned at a surface of the cantilever end effector to receive a force imparted by at least one of the second wire and a second wire anchor by contact with the at least one of the second wire and the second wire anchor, while the second actuator imparts the proximal direction force to the second wire; and
a second sensor operatively coupled to produce a second sensor signal indicative of fluid pressure within the second fluid filled sac.

19. A method to determine magnitude of a force imparted to a working jaw surface of an end effector jaw disposed at a distal end of a surgical instrument shaft, comprising:
imparting a reaction force to the end effector jaw to match the force imparted at the working jaw surface;
converting the force imparted at the working jaw surface to an increased fluid pressure within a fluid filled sac; and
converting the increased fluid pressure within a fluid filled sac to a sensor signal indicative of the increase fluid pressure.

20. A method to determine magnitude of a rotational force imparted to an end effector mounted for rotation about a pivot axis at the distal wrist portion of a surgical instrument shaft, comprising:
imparting a reaction force to a cable coupled to provide a reaction rotational force at the distal wrist portion of the surgical instrument shaft, the reaction rotational force having a magnitude to match the rotational force imparted at the end effector,
converting the reaction rotational force imparted at the distal wrist portion of the surgical instrument shaft to an increased fluid pressure within a fluid filled sac; and
converting the increased fluid pressure within a fluid filled sac to a sensor signal indicative of the increase fluid pressure.
